(19) United States
(12) Patent Application Publication
(10) Pub. No.: US 2025/0090730 A1
(43) Pub. Date: Mar. 20, 2025

(54) FLEXICOAT BLOOD-INTERFACE MATERIALS FOR BIO-COMPATABLE IMPLANTS AND DEVICES

(71) Applicant: THE UAB RESEARCH FOUNDATION, BIRMINGHAM, AL (US)

(72) Inventors: Vinoy Thomas, Hoover, AL (US); Jesse George Atherton Jones, Mountain Brook, AL (US)

(21) Appl. No.: 18/726,587

(22) PCT Filed: Jan. 3, 2023
(86) PCT No.: PCT/US2023/060037

§ 371 Date: Jul. 3, 2024

Related U.S. Application Data
(60) Provisional application No. 63/296,048, filed on Jan. 3, 2022.

(51) Int. Cl. A61L 29/10; A61L 29/12; A61L 31/08

(52) U.S. Cl. CPC A61L 29/106; A61L 29/123; A61L 31/088; A61L 2400/12; A61L 2400/18; A61L 2420/02; A61L 2420/04

(57) ABSTRACT

Various examples are provided related to blood-interface materials for metallic biomedical implants and devices. In one example, a bio-compatible implant or device includes an organosilane plasma polymerization (OPP) coating disposed on a surface of a metallic structure. The OPP coating can include inorganic silica disposed on bare metal of the metallic structure and forming a nano-textured surface. In another example, a biocompatible implant or device includes a composite coating disposed on a surface of the metallic structure. The composite coating can include silica-DEA, silica-MEA or silica-TEA coating disposed on bare metal of the metallic structure and forming a nano-textured surface. In another example, a method includes providing a metallic structure and exposing it to an OPP process to form a coating on the surface of the metallic structure to form the bio-compatible implant or device.

### CROSS REFERENCE TO RELATED APPLICATIONS

This application claims priority to, and the benefit of, co-pending U.S. provisional application entitled “Flexicoat Blood-Interface Materials for Metallic Implants and Stent Devices” having Ser. No. 63/296,048, filed Jan. 3, 2021, which is hereby incorporated by reference in its entirety.

### BACKGROUND

Stroke is a cardiometabolic disease that disproportionately affects minority African American, Hispanic, and Asian communities. Unique genetic, biologic, and environmental factors predispose these populations to intracranial atherosclerosis (ICAD) and subsequent stroke. Race, geography, socioeconomic and environmental factors all contribute to health disparities in clinical outcome, with black to white mortality ratio of 4.0. Moreover, current treatment of ICAD has shown poor outcomes relative to atrial fibrillation in the acute stroke setting. Such factors have left these communities underserved by the healthcare system.

### SUMMARY

Aspects of the present disclosure are related to blood-interface materials for biomedical implants and devices. In one aspect, among others, a bio-compatible implant or device comprises a metallic structure and an organosilane plasma polymerization (OPP) coating disposed on a surface of the metallic structure, the OPP coating comprising inorganic silica disposed on bare metal of the metallic structure and forming a nano-textured surface. In one or more aspects, the metallic structure can be a stent, wire/guidewire or catheter. The metallic structure can comprise nitinol (NiTi), stainless steel, titanium, tungsten, platinum, cobalt, chromium or cobalt chrome alloy. In various aspects, the OPP coating can be a SiOₓ-like coating disposed on the surface of the metallic structure. The OPP coating can be disposed on the surface by plasma treatment over a period of approximately 30 minutes. The surface of the metallic structure can be pretreated prior to the plasma treatment. The OPP coating can have a thickness in a range from about 50 nm to about 1 micron.

In another aspect, a bio-compatible implant or device comprises a metallic structure and a composite coating disposed on a surface of the metallic structure, the composite coating comprising silica-DEA, silica-MEA, or silica-TEA functionalized coating disposed on bare metal of the metallic structure and forming a nano-textured surface. In one or more aspects, the composite coating can comprise silica-DEA, dopamine, dopamine acrylic derivatives or dopamine acrylamide polymerized/co-polymerized coating disposed on the bare metal of the metallic structure. The composite coating can comprise silica-allyl amine, L-lysine, or tyrosine, polymerized/co-polymerized coating disposed on the bare metal of the metallic structure. The metallic structure can be a stent, wire/guidewire or catheter. The metallic structure can comprise nitinol (NiTi), stainless steel, titanium, tungsten, platinum, cobalt, chromium or cobalt chrome alloy. In various aspects, the composite coating can be disposed on the surface by plasma treatment, which can be over a period of approximately 30 minutes. The surface of the metallic structure can be pretreated prior to the plasma treatment. The composite coating can have a thickness in a range from about 50 nm to about 1 micron.

In another aspect, among others, a bio-compatible implant or device comprises a non-metallic or polymeric structure and an organosilane plasma polymerization (OPP) coating disposed on a surface of the non-metallic or polymeric structure, the OPP coating comprising inorganic silica disposed on bare surface of the non-metallic or polymeric structure and forming a nano-textured surface. In one or more aspects, the non-metallic or polymeric structure can be a stent, wire/guidewire or catheter. The non-metallic or polymeric structure can comprise polytetrafluoroethylene (PTFE) or polyethylene terephthalate (PET). In various aspects, the OPP coating can be a SiOₓ-like coating disposed on the surface of the non-metallic or polymeric structure. The OPP coating can be disposed on the surface by plasma treatment over a period of approximately 30 minutes. The surface of the non-metallic or polymeric structure can be pretreated prior to the plasma treatment. The OPP coating can have a thickness in a range from about 50 nm to about 1 micron.

In another aspect, a bio-compatible implant or device comprises a non-metallic or polymeric structure and a composite coating disposed on a surface of the non-metallic or polymeric structure, the composite coating comprising silica-DEA, silica-MEA, or silica-TEA functionalized coating disposed on bare surface of the non-metallic or polymeric structure and forming a nano-textured surface. In one or more aspects, the composite coating can comprise silica-DEA, dopamine, dopamine acrylic derivatives or dopamine acrylamide polymerized/co-polymerized coating disposed on the bare surface of the non-metallic or polymeric structure. The composite coating can comprise silica-allyl amine, L-lysine, or tyrosine, polymerized/co-polymerized coating disposed on the bare surface of the non-metallic or polymeric structure. The non-metallic or polymeric structure can be a stent, wire/guidewire or catheter. The non-metallic or polymeric structure can comprise nitinol (NiTi), stainless steel, titanium, tungsten, platinum, cobalt, chromium or cobalt chrome alloy. In various aspects, the composite coating can be disposed on the surface by plasma treatment, which can be over a period of approximately 30 minutes. The surface of the non-metallic or polymeric structure can be pretreated prior to the plasma treatment. The composite coating can have a thickness in a range from about 50 nm to about 1 micron.

In another aspect, a method for preparing a bio-compatible implant or device comprises providing a metallic, non-metallic or polymeric structure; and exposing the metallic, non-metallic or polymeric structure to organosilane plasma polymerization (OPP) process to form an OPP coating on a surface of the metallic, non-metallic or polymeric structure to form the bio-compatible implant or device. The OPP coating can comprise inorganic silica disposed on bare metal of the metallic structure thereby forming a nano-textured surface. In one or more aspects, the OPP coating can be a composite coating comprising silica-DEA, silica-MEA, or silica-TEA functionalized coating. The OPP coating can be disposed on the surface by plasma treatment over a period of approximately 30 minutes. The OPP coating can have a thickness in a range from about 50 nm to about 1 micron.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 Illustrates examples of live/dead endothelial cell assays on PTFE.

FIG. 2 is schematic representation illustrating a silane plasma polymerization process taking place on the surface of PTFE.

FIG. 3 illustrates examples of nanoscale surface roughness and absence of exposed metal from organosilane plasma polymerization (OPP), in accordance with various embodiments of the present disclosure.

FIGS. 4A-4C illustrate effects of OPP treatment of a nitinol (NiTi) stent, in accordance with various embodiments of the present disclosure.

FIG. 5 is an image of an example of a Harrick system, in accordance with various embodiments of the present disclosure.

FIG. 6 illustrates examples of pressure data for precursors MEA (mono-ethanolamine), DEA (di-ethanolamine), and TEA (tri-ethanolamine), in accordance with various embodiments of the present disclosure.

FIG. 7 illustrates examples of temperature profiles, in accordance with various embodiments of the present disclosure.

FIG. 8 illustrates examples of compiled contact angle data, in accordance with various embodiments of the present disclosure.

FIG. 9 illustrates examples of BSA-FITC (bovine serum albumin-fluorescein isothiocyanate modified) adsorption tests, in accordance with various embodiments of the present disclosure.

FIG. 10A illustrates examples of spot test for NO release using a Griess assay, in accordance with various embodiments of the present disclosure.

FIG. 10B is a scanning electron microscope (SEM) image showing an example of SEM of an implant composite coating silica-DEA coating, in accordance with various embodiments of the present disclosure.

### DETAILED DESCRIPTION

Disclosed herein are various examples related to blood-interface materials for biomedical implants and devices. A super-hydrophilic durable bio-interface surface is disclosed for biomedical devices such as, e.g., stents, guidewires/wires and catheters. The bio-interface material can assist in the reduction of blood clots on the material surface, enhance endothelial cell growth on the material surface, prevent metal ion leaching, reduce friction between the vessel wall and/or luminal contents and material surface. This may also be applied to wholly or partially polymeric (e.g., PTFE or PET) devices such as catheters. Herein, the bio-interface material can reduce friction between the blood vessel wall and outer catheter or wire surface to facilitate intravascular navigation. For aspiration catheters, the bio-interface material can reduce friction between blood clots and other intraluminal contents and inner catheter surface to facilitate ingestion. Examples of other biomedical devices include, but are not limited to, temporary catheters (e.g., silicone, polyurethane), biostable PTFE tubular dialysis access graft and biostable PET tubular graft for dialysis, or biodegrable/template conduits for nerve/spinal cord. Reference will now be made in detail to the description of the embodiments as illustrated in the drawings, wherein like reference numbers indicate like parts throughout the several views.

Intravascular stenting has been shown to effectively restore arterial and venous patency by facilitating wider luminal diameter for free-flowing blood segregated from thrombogenic plaque, clot, and fibrous tissue among other lesions. Outward radial force and interstitial cell size are mechanical variables in determining successful stent assisted recanalization. However, biologic response to stenting is conversely unfavorable and includes immune mediated foreign body reaction, platelet activation, and neointimal hyperplasia. Existing strategies to overcome these obstacles have focused on concomitant anti-platelet and anti-mitotic medical management but do not address the fundamental bio-incompatibility of current devices. Anti-platelet medications such as aspirin and clopidogrel (DAPT) lower, but do not eliminate, the rate of thrombosis. Moreover, DAPT in acute ischemic stroke is associated with 2.5-increased relative risk of brain hemorrhage, which limits the utilization and safety of stenting.

Quantitative characterization of a sufficiently biocompatible vascular stent that can be successfully implanted without DAPT is presented. While the discussion is presented in the context of stents, the methodology is equally applicable to other biomedical implants and devices. Prior studies initially pointed to heparin and paclitaxel drug eluting stents, which demonstrated early positive results yet eventually failed as their therapeutic effect diminished prior to adequate endothelial migration and maturation. The scientific knowledge of stent-induced biological changes can be improved by investigating platelet and endothelial response to a novel nano-textured stent. Nanoscale surface roughness has been shown to repel platelets through enhanced wettability, i.e., super-hydrophilic property. Endothelial cell migration can be facilitated along linear etched grooves in nano-textured polytetrafluoroethylene (PTFE) graft material. Deposition of an organosilane layer on the surface of PTFE was found to provide favorable surface properties to the PTFE such as a very high surface oxygen content, high hydrophilicity and improved surface mechanics. This was found to facilitate rapid cell growth and decreased platelet attachment.

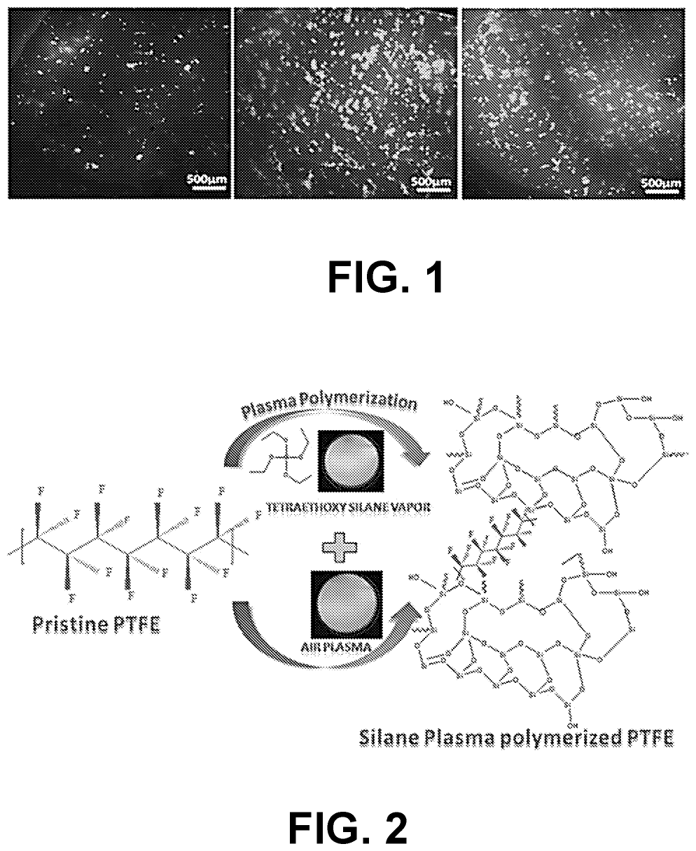

Plasma polymerization is a phenomenon in which vapors of an organic monomer undergo a series of chemical reactions in the plasma phase such as hydrolysis and condensation and get polymerized. TEOS is one such monomer which can undergo plasma polymerization via the hydrolysis and condensation reactions. The silica polymerization is usually accomplished via a sol-gel reaction in wet chemistry methods. The plasma polymerization capability of TEOS was used to polymerize and modify the surface properties of PTFE. Air plasma was combined with the vapors of TEOS to facilitate the hydrolysis and condensation reactions to form a plasma polymerized silane coating over the surface of PTFE. FIG. 1 includes images of live/dead endothelial cell assay on pristine PTFE in the left image, PTFE-t10 (10-minute OPP application) in the center image, and PTFE-t20 surfaces after 3 days of cell seeding in the right image. The platelet adhesion studies suggest the potential non thrombogenicity of the PTFE-t10 and PTFE-t20 surfaces. Additional information can be found in “Non-equilibrium organosilane plasma polymerization for modulating the surface of PTFE towards potential blood contact applications” by V. M. Vihayan et al. (J. Mater. Chem. B, 2020, 8, 2814), which is hereby incorporated by reference in its entirety, is outlined below.

Surface modification can augment biomaterials for appropriate cell responses. Plasma treatment and/or polymerization is a facile surface modification technique for polymers that has been employed for decades. The nondestructive and in situ sterilization capabilities of this technique make it an attractive candidate for modifying the surface properties of biomaterials without compromising their bulk properties. Plasma, the fourth state of matter, is composed of mixtures of ions, electrons, radicals, and neutral atoms and/or molecules which, upon colliding on the surface of materials, can rearrange or alter their surface chemistry. It can introduce various surface functional groups such as amino, carboxyl and hydroxyl groups on their surface.

These functional groups can be further conjugated with various biomolecules, growth factors or peptides for a variety of biomedical applications. The surface properties of biomaterials determine the protein and/or cellular responses which in turn will decide the success rate of implant biomaterials inside the body. Chemical surface modification can be accomplished through performing certain surface reactions by wet chemistry. This process is time consuming and can also lead to some residual chemicals over the surface. This can affect the functional performance of a material inside the body. The absence of multiple reagents that wet chemistry use for surface functionalization, thus, makes plasma treatment a safe alternative method for surface modification of biomaterials.

Plasma surface modification is a simple and robust method that can safely and reliably modify the surface properties of biomaterials towards different biomedical applications. However, the plasma surface modification of biomaterials is typically accomplished using conventional feed gases such as oxygen, ammonia, nitrogen and hydrogen. These gases can introduce different functional groups such as carboxyl, amino and hydroxyl groups. However, these conventionally modified surfaces are always subject to ageing (surface reorganization); thus, ageing hinders their long-term ability to retain the material properties associated with better cellular responses.

Plasma can induce polymerization of volatile organic monomers through a process called plasma enhanced chemical vapor deposition (PECVD), where the polymers can be deposited over the surface of a substrate. The high energy species formed as a result of this process can cause a chain of reaction and, subsequently, cause the polymerization of the reactive monomers. But, unlike conventional polymers, plasma-based polymers are not well organized as they have a random arrangement. Plasma polymerization can play a role in tissue regeneration applications. Plasma polymerization of reactive monomers can tailor the surface properties of metallic and polymeric biomaterials to endow them with favorable cellular responses. More specific examples include plasma polymerization of organic monomers like acrylic acid and allyl amine on metallic or polymeric biomaterials. Results of these studies suggest that these organic monomers, when plasma polymerized, endow polymer biomaterials with better cell adhesion and proliferation capabilities. More importantly, stability studies conducted on plasma polymerized acrylic acid coatings have exhibited high stability, suggesting their potential utility for different biomedical applications. Hence, plasma polymerizations of organic monomers have a wide scope to tailor the surface properties of biomaterials for different bio-interface applications.

PTFE is a fluoropolymer which is widely used as a vascular graft material. The chemically inert nature of PTFE makes it an ideal implantable material. Even though large diameter PTFE vascular grafts (≥6 mm) have been reported as successful, small diameter PTFE vascular grafts (≤4 mm) still have serious issues. Challenges associated with small diameter PTFE vascular grafts include thrombosis and lack of endothelial cell growth. The hydrophobic nature of PTFE makes it very difficult for endothelial cells to attach and grow to a confluent layer. Hence, the surface properties of PTFE can be tailored to meet the requirements of small diameter vascular grafts.

One method of modifying the surface properties of PTFE is plasma modification. Different types of plasma processing can be used for modifying the surface properties of PTFE. Most include oxygen plasma, ammonia plasma and hydrogen plasma processing. The major drawback of these plasma surface modification routes is ageing (a significant reduction in functional groups with time). Moreover, postprocessing multistep conjugations with peptides and antithrombotic agents are further needed to favor endothelial cell growth. Recently, hybrid processes (plasma modification and chemical modification) have been reported to tailor the surface properties of PTFE for blood contact applications. These processes utilized the combination of oxygen plasma and dopamine surface functionalization for improving the endothelial cell affinity and anti-thrombogenicity. However, these types of hybrid processes use multiple chemical reagents with several steps which are time consuming. Hence, a more efficient and facile method of surface modification of PTFE would be beneficial for blood contact applications.

Plasma polymerizations of organic monomers has never been explored to tailor the surface properties of PTFE for blood contact applications. Inspired by this idea, plasma polymerization of an organosilane precursor has been explored, more specifically tertraethoxysilane (TEOS) to modify the surface properties of PTFE for blood contact applications. The plasma polymerization of TEOS may endow PTFE with favorable surface properties for potential blood contact applications. The plasma polymerization capability of this organosilane monomer for tailoring/modifying the surface properties of PTFE has been explored for blood contact applications.

The PTFE substrate used for the plasma modification (Laboratory grade PTFE sheets) was purchased from Oil sleek company, USA. The Harrick Plasma chamber (PDC-001-HP) used for the plasma surface modification was purchased from Harrick Plasma, New York, USA. The reagents used for the experiments such as tetraethoxysilane and acetone were purchased from Sigma Aldrich.

Plasma polymerization of tertraethoxysilane on PTFE. The PTFE sheets were cut into 3 cm×1.2 cm (0.2 mm thickness) pieces for plasma treatment. Briefly, the samples were washed with acetone for 30 min before the plasma treatment to remove the adsorbed impurities (if any) from the surface. The PTFE samples were then placed inside a Harrick Plasma chamber (PDC-001-HP) and a radiofrequency (13.56 MHz, 45 W) was used for plasma treatment. The plasma polymerization process of TEOS was accomplished by using a combination of a TEOS-air system inside the plasma chamber. Briefly, 1 mL of TEOS was placed on a glass slide adjacent to the PTFE samples inside the chamber, followed by applying a constant Air flow rate of 50 sccm inside the chamber. The reduced pressure (500 mTorr inside the chamber) facilitates the formation of TEOS vapors. Different plasma treatment times such as 10, 20 and 30 minutes were employed for optimizing the plasma polymerization process. Herein they are referred to as PTFE-t10, PTFE-t20 and PTFE-t30 which correspond to 10 min, 20 min, and 30 min respectively.

Characterization. Fourier-transform infrared spectroscopy (FTIR) and X-ray photoelectron spectroscopy (XPS) were employed to elucidate the surface chemistry. The Bruker alpha FTIR spectrometer with ATR mode was used to acquire IR-absorption spectra (ranging from 4000 to 400 cm_1). The XPS spectra of plasma treated samples were obtained using a Phi 5000 Versaprobe made by Phi Electronics, Inc. (Chanhassen, WI USA). The X-ray source of this instrument is a monochromatic, focused, AI K-alpha source (E=1486.6 eV) at 25 W with a 100 micrometer spot size. A Mg anode (I=1253.6 eV) was used at 300 W and a barium oxide neutralizer eliminated the charging. The survey scans (4 scans averaged per analysis) were obtained using a pass energy of 187.5 eV with a step size of 0.5 eV. The high resolution scans (8 scans average per analysis) were obtained with a pass energy of 23.5 eV and a step size of 0.1 eV.

To measure the contact angle, the samples (n=3) were mounted onto a glass slide. Contact angles were measured using a sessile drop method at room temperature. The water droplet size was 5 mL. ImageJ software was used to accurately measure the contact angle of the water droplets on the surface.

The X-ray diffraction (XRD) experiments were performed on an Empyrean X-ray diffractometer (Malvern Panalytical, UK) equipped with a Cu LFF HR X-ray tube at 30 kV tension and 10 mA current. The spectrum was recorded in the range of 2y from 10 to 100. The structure and morphology of the plasma treated and untreated control PTFE tape were characterized by scanning electron microscopy (SEM) after sputter-coated with Au—Pd and observed using a FE-SEM (Quanta FEG 650 from FEI, Hillsboro, OR) and images were taken at different magnifications.

Hardness and Young's modulus were measured using an MTS NanoIndenter XP having a Berkovich diamond tip with a nominal radius of 50 nm. Tip calibration was performed on the fused silica standard (an accepted Young's modulus of 72 GPa) before and after testing all PTFE samples. All indents, including those on silica, were made to a maximum load of 1.5 mN. The measured Young's modulus and hardness values were determined at the maximum load. Young's modulus of the silica before and after testing the PTFE surfaces was 71.9 _ 1.0 GPa and 72.0 _ 3.0 GPa, respectively. Therefore, moduli from the silica standard did not vary by more than 6%. 15 indents were made on each sample for statistical analysis.

Cell culture conditions. Human aortic endothelial cells (HAECs) were purchased from Lonza, Inc and cultured in Endothelial Growth Media (EGM-2 BulletKit; Lonza, Walkersville, MD). HAECs were grown to 70-80% confluence under normal cell culture conditions (37 1C, 95% humidity, 5% CO2) before being seeded onto PTFE sheets.

MTS assay on PTFE sheets. Samples were prepared by cutting PTFE sheets into circles with diameters of 6.4 mm with various durations of plasma treatment and then sterilizing them with UV light for 3 hours. The sterile samples were then placed into a 96-well plate. 9000 HAEC cells in 200 mL of media were seeded onto each sheet and cultured in an incubator at 37 1C. After culture, an MTS [3-(4,5-dimethylthiazol-2-yl)-5-(3-carboxymethoxyphenyl)-2-(4-sulfophenyl)-2H-tetrazolium] assay (CellTiter 96 solution, Promega Co.) was performed to quantify HAEC proliferation on the sheets at 1, 3, and 5 days. HAEC proliferation was assessed on 5 PTFE sheets for each duration of plasma treatment.

Live/dead assay on PTFE sheets. Samples were prepared by cutting the PTFE sheets into circles with diameters of 9.5 mm and then they were UV sterilized for 3 hours. The samples were then placed into 48-well plates and 25 000 HAEC cells in 400 mL of media were seeded onto each sheet. The cells were cultured at 37° C. for 3 days. After 3 days of culture, viable cells on the sheets were stained by conducting a live/dead viability assay (Molecular Probes Inc., OR). Stained cells were imaged using a Nikon fluorescent microscope and ImageJ software.

Human platelet adhesion on PTFE sheets. Samples were prepared similarly to those prepared for the live/dead assay and placed in 48-well plates. Platelets (Innovative Research, Inc.) were diluted with Tyrode's solution to a concentration of 6×10⁸ platelets per mL. Platelets were then seeded onto the sheets and allowed to incubate for 30 minutes. The sheets were then removed from the plate and washed with PBS to remove free floating platelets. After staining the sheets with calcein AM solution, platelets were visualized with a Nikon fluorescent microscope and ImageJ software.

SEM imaging for PTFE sheets. Samples were prepared similarly to those prepared for the live/dead assay and placed in 48-well plates. 25 000 HAEC cells in 400 mL of media were seeded onto each sheet and the cells were cultured at 37° C. for 3 days. After culture, the cells were fixed with paraformaldehyde. The fixed samples were dehydrated with ethanol. The PTFE sheets were imaged using a QuantaTM 650 FEG (FEI Co.) with an accelerating voltage of 10 kV.

Cytoskeletal staining. Samples of the material were prepared, and cells were cultured as described for the live/dead analysis. Post culture samples were washed with PBS (1×, 5 min); fixed with paraformaldehyde (4%, 20 min); washed with additional PBS (1×, 5 min), adding Triton X-100 (0.1%). Staining was conducted with 200 mL of the staining solution (PBS 1×, BSA 1%, DAPI 0.1 mg mL⁻¹), the phalloidin-rhodamine conjugate (Abcam, 1× conc.), under dark conditions for 40 min. Then, the samples were rinsed with PBS (1×, 5 min) and the cover slips were added before imaging with a Nikon fluorescent microscope and ImageJ to process the data.

Statistical analysis. The number of specimens tested for each group was 5 (n=5). The obtained data were tested for statistical significance using the ANOVA method (Using the GraphPad Prism software) and p≤0.05 was defined as significant.

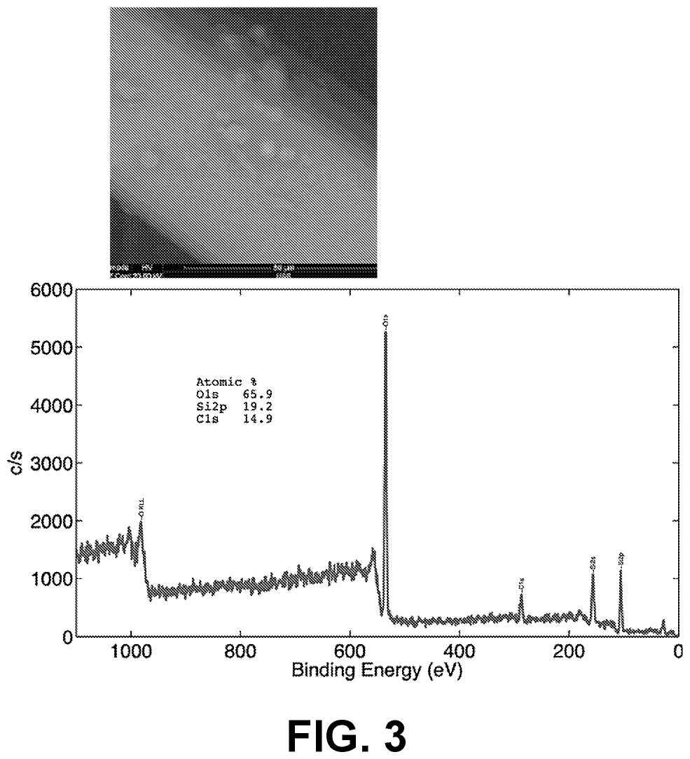

Plasma polymerization is a phenomenon in which vapors of an organic monomer undergo a series of chemical reactions in the plasma phase such as hydrolysis and condensation and get polymerized. TEOS is one such monomer which can undergo plasma polymerization via the hydrolysis and condensation reactions. The silica polymerization is usually accomplished via a sol-gel reaction in wet chemistry methods. However, plasma-based polymerization does not require the use of any bases, solvents and high temperature. Hence, it is a far greener method in comparison with the conventional sol-gel method. The plasma polymerization capability of TEOS was used to polymerize and modify the surface properties of PTFE, which is a widely used vascular graft material. Air plasma was combined with the vapors of TEOS to facilitate the necessary hydrolysis and condensation reactions to form a plasma polymerized silane coating over the surface of PTFE (Scheme 1), shown in FIG. 2.

Results and discussion can be found in “Non-equilibrium organosilane plasma polymerization for modulating the surface of PTFE towards potential blood contact applications” by V. M. Vihayan et al. (J. Mater. Chem. B, 2020, 8, 2814), which is hereby incorporated by reference in its entirety.

This material science concept of surface interactions can be extended to apply to acute ischemic stroke with the goal of improving health through more successful reperfusion therapies. Several methods can be employed to produce gradations (e.g., 100 nm gradations) in the vertical depth of the metallic surface to induce super-hydrophilic properties. For example, surface processing can include, but is not limited to, femtosecond laser, chemical etching, and polymer adhesion. Unfortunately, fabricating a durable and uniform nanoscale surface over a complex three-dimensional implant such as, e.g., a stent mask has proven challenging to accomplish at feasible cost.

However, test data has revealed that organosilane plasma polymerization (OPP) of inorganic silica onto bare metal such as, e.g., nitinol (NiTi), stainless steel, titanium, tungsten, platinum, cobalt, chromium or cobalt chrome alloy stents or other biomedical implants or devices 1) distributes a uniform, nano-textured surface, 2) produces a highly stable surface resistant to fracture, shearing or reorganization, and 3) prevents nickel leaching from nitinol moieties. These novel and advanced nano-textured stents can be used for modulation of platelet and endothelial response to therapy. OPP can be utilized to fabricate a uniform nano-textured surface across modified bare metal stents or other biomedical implants or devices. A nano-textured surface can include regular or irregular variations in the surface at a submicron level. For example, the variations can be in a range from about 10 nm to about 900 nm, from about 10 nm to about 800 nm, from about 10 nm to about 750 nm, from about 10 nm to about 650 nm, or from about 10 nm to about 500 nm, or other submicron range. Furthermore, it has be found that applying OPP to stents by immersion at room temperature is more cost effective relative to laser etching or phosphorylcholine polymer preparation.

OPP can be used for surface modification of biomedical implants and devices including metallic and non-metallic (e.g., polymeric) structures. In this technique, organic precursor monomers deposit and polymerize on a surface of the metallic structure through plasma processing. The reduced pressure and the presence of radio frequency (RF) energy inside the plasma chamber cause the monomer to ionize, and subsequently polymerize, resulting in surface deposition. OPP coatings based on acrylate monomers (MMA) can be used for modifying the surface properties of biomaterials to be favorable for cell growth, proliferation and differentiation. A hybrid process which combines the etching capability of oxygen plasma (top-down approach) with the plasma polymerization capability of hydrophilic monomers (bottom-up approach) such as silane precursors can be iterated to coat a super hydrophilic surface onto metallic implants and devices such as, e.g., nitinol or other metallic stents. The process can also be applied to non-metallic (e.g., polymeric) implants and devices. The thickness of the OPP coating can be in a range from, e.g., about 50 nm to about 1 micron or greater, about 100 nm or greater, about 200 nm or greater, or about 250 nm or greater. The low temperature OPP can improve the surface characteristics of the metal (e.g., nickel, titanium, nitinol (NiTi), stainless steel, titanium, tungsten, platinum, cobalt chrome alloys, etc.) at lower RF power and shorter timescales than previously reported. The polymerized silane coating can impart a highly adhesive surface for endothelial cell proliferation due to the preponderance of surface hydroxyl groups, while large polymer networks formed over the surface provide steric hindrance to platelet adhesion.

OPP modified stents can enable stent implantation in the acute stroke setting with reduced thrombotic and hemorrhagic complications. This could change clinical practice with respect to acute stroke, as more frequent indicated use of intracranial and cervical carotid or vertebral, etc. stents drives higher Thrombolysis in Cerebral Infarction (TICI) scores, and ultimately a greater proportion of patients achieving<modified Rankin scale (mRS) 3 outcome at 90 days. For example, implanted nano-textured stents or other biomedical implants or devices modified by OPP can offer improved biocompatibility relative to existing devices.

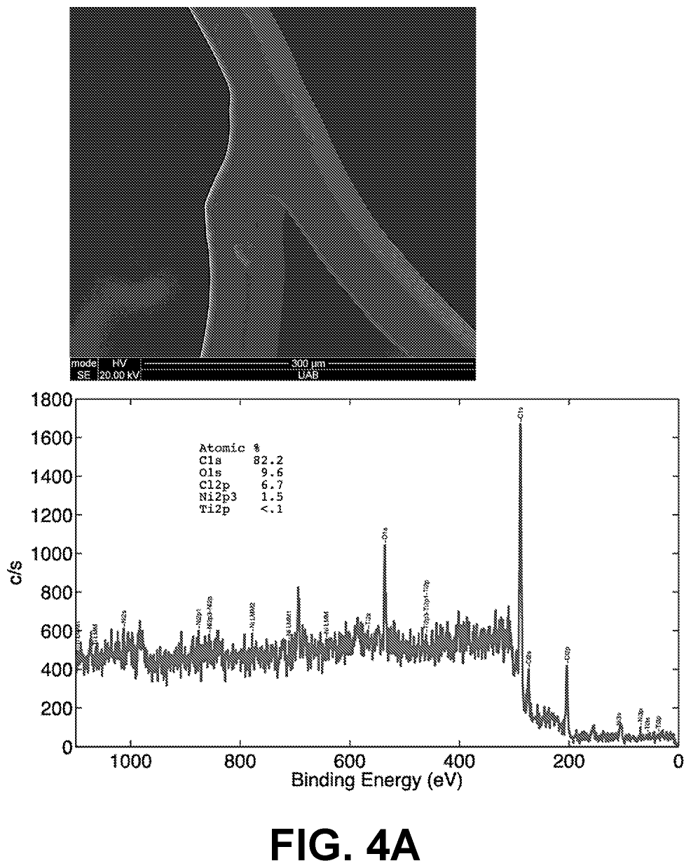

Self-expanding bare metal stents restore luminal caliber in stenosed or occluded vessels through outward radial force. As the stent is deployed, its tines appose the endothelial surface ultimately leading to a patent channel for anterograde blood flow. A preliminary proof-of-concept design was implemented for an open cell nitinol stent. An optimal plasma exposure time point of 30 minutes was considered. FIG. 3 includes electron microscopy (left pane) and mass spectroscopy (right pane) that demonstrate the resulting nanoscale surface roughness and absence of exposed nickel after being modified by a 30-minute organosilane immersion.

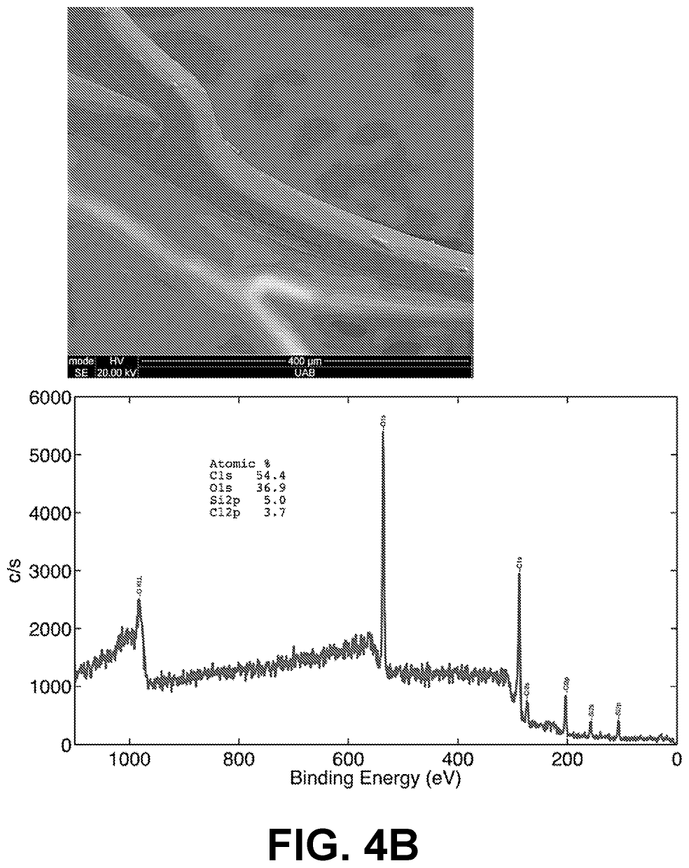

This is further demonstrated in FIG. 4A-4C. FIG. 4A includes a scanning electron microscope (SEM) image (top pane) and X-ray photoelectron spectroscopy (XPS) (bottom pane) of a nitinol stent. Ni and Ti on the surface can be seen on the control. A superhydrophobic surface (water contact ang above 150 degrees) which can roll-off water and resist protein absorption can be formed on a nitinol stent by treated with methyl methacrylate (MMA) in plasma, e.g., for 1 hour. FIG. 4B includes a SEM image (top pane) and XPS (bottom pane) of the hydrophobic MMA treated nitinol stent. The OPP method can also be applied to from a hydrophilic OPP treated nitinol stent.

FIG. 4C includes a SEM images (top panes) and XPS (bottom pane) of the hydrophilic OPP treated nitinol stent. The top left pane shows a modified stent treated for 10 minutes. The surface morphology of the modified stent is the same as the control. The top right pane shows the uniform surface of a SiOₓ coated stent resulting from plasma treatment for a total of 30 minutes. As indicated by the XPS, the effectiveness of the SiOₓ-like coating is illustrated by the absence of the Ti and Ni peaks and the increased Si peak in FIG. 4C. SiOₓ-like coatings can include, e.g., a silica composite coating (e.g., SiOₓ, where x is 2 or greater, or SiOₓ is a polymerized silica) functionalized with amines (e.g., MEA, DEA, TEA, allylamine, tyrosine, or dopamine, or their derivates). The composite coating can comprise a silica-DEA, silica-MEA, or silica-TEA functionalized coating disposed on bare metal of the metallic structure and forming a nano-textured surface. For example, the composite coating can comprise silica DEA, dopamine, dopamine acrylic derivatives (e.g., dopamine acrylate or dopamine acrylic acid), or dopamine acrylamide polymerized/co-polymerized coating disposed on bare metal. The composite coating can comprise silica-allyl amine, L-lysine, or tyrosine, polymerized/co-polymerized coating disposed on the bare metal.

Advantages of the hydrophilic OPP treated surfaces over hydrophobic surfaces can include:
Hydrophilic glass-like surfaces are thermodynamically compatible with aqueous media (e.g., blood) and can be stable.
Stable coating can prevent Ni leaching.
The surface functional group (—NH2, OH, etc.) can be altered to have better cell attachment.
Hardness can provide scratch resistance.
Short process time.

A smooth nano surface can be created on the surface of the metallic structure by pretreating before the OPP process. The smooth surface can enable frictionless movement of the stent in the catheter. The OPP treatment can also prevent Ni migration to the surface as seen with laser methods. The plasma coating can also prevent Ni ion leaching while not changing the flexible mechanical properties of the stent (or metallic implant).

Nanoscale surface quantification of the modified devices can involve systematic ex vivo characterization via analytical techniques such as, e.g., Fourier transform infrared spectroscopy (FTIR), X-ray photoelectron spectroscopy (XPS), X-ray diffractometer (XRD), confocal laser microscopy, scanning electron microscopy (SEM), and/or atomic force microscopy (AFM). Previous studies have demonstrated successful plasma polymerization and deposition on a substrate surface.

### Plasma Processing for Silica Composite Coating With Amines (MEA, DEA, TEA, Allylamine, Tyrosine, or Dopamine and Their Derivates)

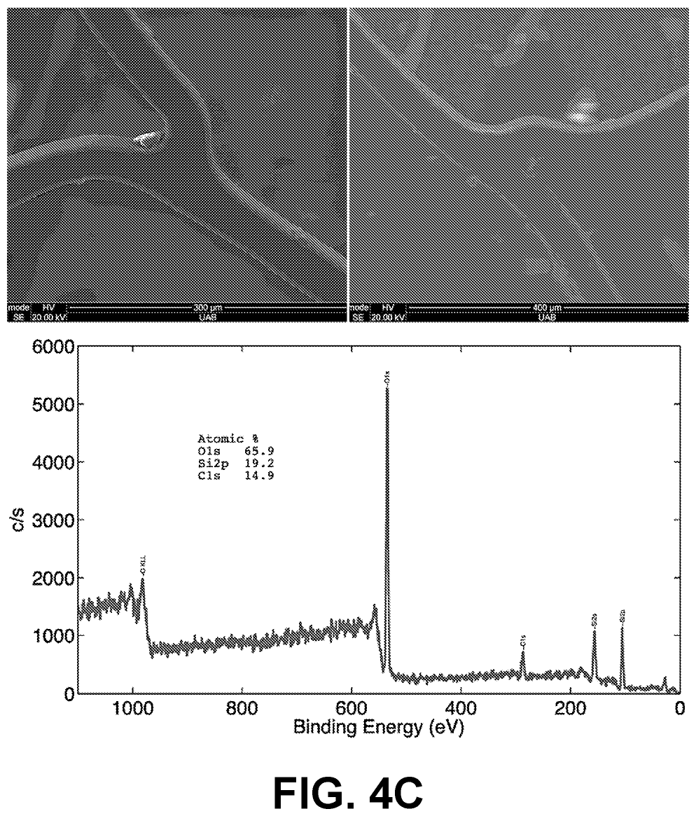

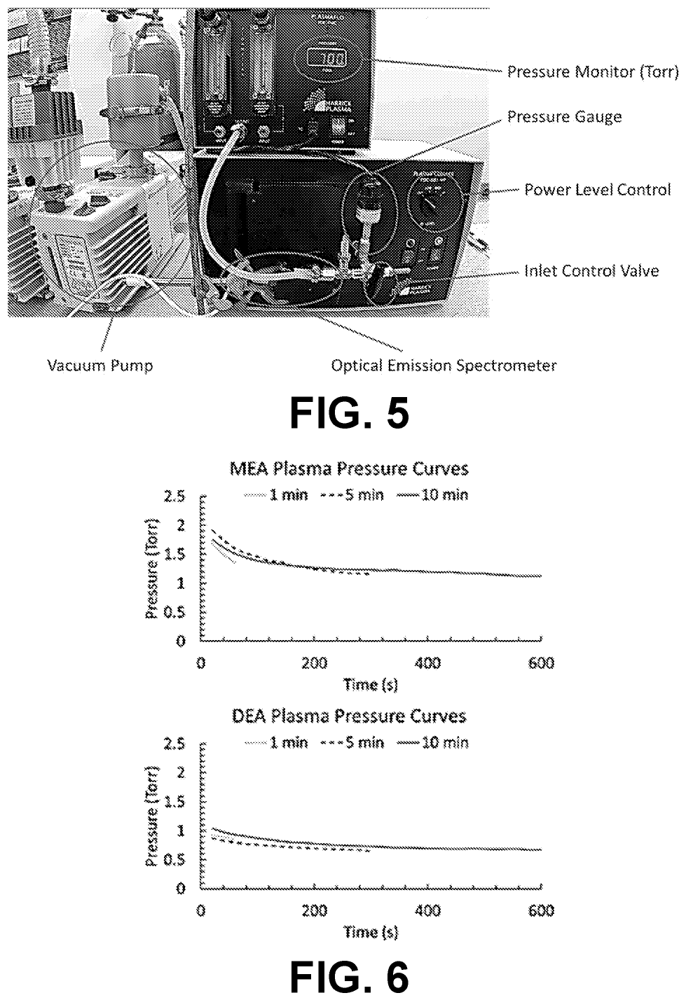

Plasma conditions were accessed with a Harrick PDC-001 plasma cleaner (Harrick Plasma, NY, USA). The 45 W setting was employed, and no feed gas used. The organic precursors were added in 100 μL increments per run with the use of a 96-well plate which also served as a sample holder. During evacuation with a pump the pressure was recorded during processing. Only the evaporation rate of the precursor controlled the mass flow in the system. FIG. 5 is an image showing the Harrick system and FIG. 6 shows the pressure data for each of the precursors MEA, DEA, and TEA. For optical emission measurements an Ocean Optics USB 4000 spectrometer (Florida, USA) was employed by tilting the optical slit toward the window of the Harrick chamber by clamping in a ring stand; positioning was marked with tape to repeat the location.

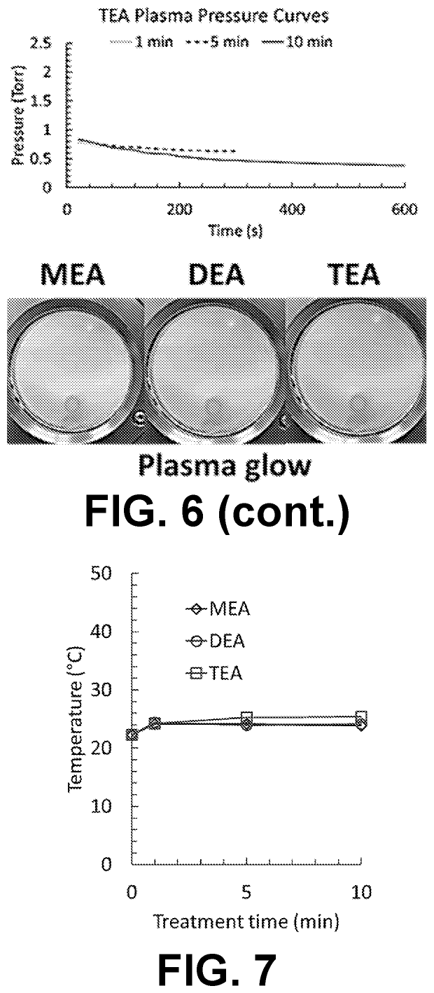

OceanSuite software was used to capture live spectra with a 500 ms integration time, 4 scan averaging, value of 2 for the boxcar average width, and electronic dark correction and nonlinear correction added. The live output was subtracted from a dark spectrum to correct for baseline. The spectrometer was uncooled and used at ambient conditions. Immediately after processing, samples were taken for temperature measurements with a FLIR C2 imager (Teledyne FLIR LLC, Oregon, USA). These images were taken against a cutting mat as a background with the optical image overlaid for additional detail. FIG. 7 shows the temperature profiles.

Contact angle experiment. For the aging and surface analysis study, a custom-built contact angle goniometer was constructed. It used a machined aluminum optical table (4″×4″) that served as the sample stage. The measurements were taken with a Canon TV zoom lens 12-75 mm with the shutter at f/16 and a +10-macro attachment added. A 5 μL drop of DI water was added with a calibrated syringe and images taken by CCD sensor attached to a 2012 MacBook Pro running USB Pluggable Digital Viewer microscope software. Angles were taken from the angle tool in ImageJ software. Aging was carried out by keeping samples at various conditions in the freezer, benchtop, and a warming plate. The samples all were kept inside 12-well plates to keep out contamination.

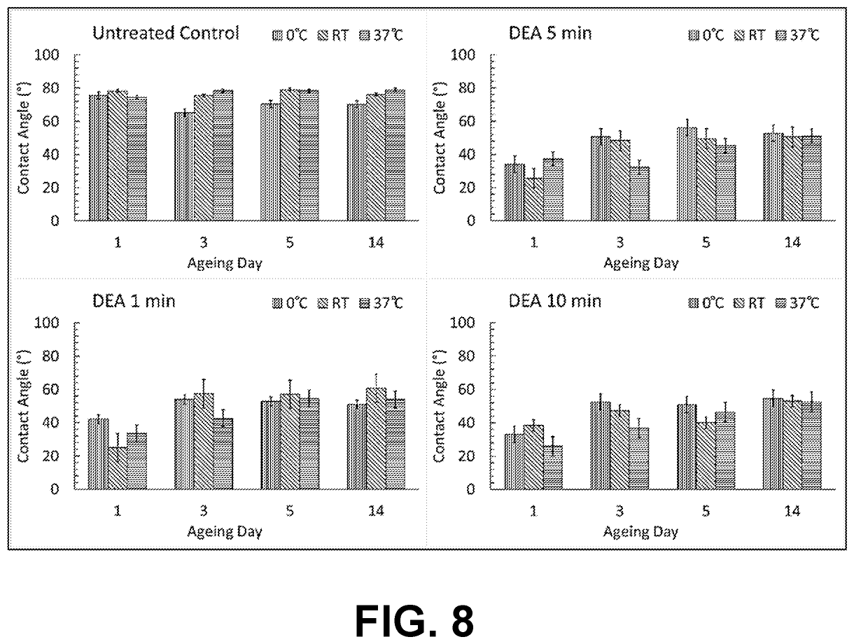

FIG. 8 illustrates examples of compiled contact angle data of an untreated control (top left) and various DEA-LTP treated samples (DEA 1 minute—bottom left, DEA 5 minutes—top right, and DEA 10 minutes—bottom right) as an evolution in time and temperature. The time points of 10 seconds, 1 minute, 5 minutes, and 10 minutes are the various LTP treatment times. Within the graphs are ageing times of 1, 3, 5, and 14 days. Bars are the standard error for 5 samples yielding 2 measurements each for n=10 per group and the values plotted are the numerical average.

Bovine Serum Albumin-fluorescein isothiocyanate modified (BSA-FITC) assay. A protein adsorption assay was conducted by taking biopsy punch samples of the materials and soaking them in 1.0 mg/mL solutions of BSA-FITC in 1× PBS buffer (7.4 pH) at RT for 12 h. Subsequently, the samples were rinsed for 12 h at RT in 1× PBS and the resultant solutions analyzed in a gel-doc imaging system with a Canon Rebel SLT camera equipped with a 28-80 mm lens set to 50 mm with f/11 at 2.5 s shutter speed or adjusted according to the onboard light meter. The images were processed in ImageJ and data collected from the output tables for line profiles to measure the intensity as a function of fluorescent intensity.

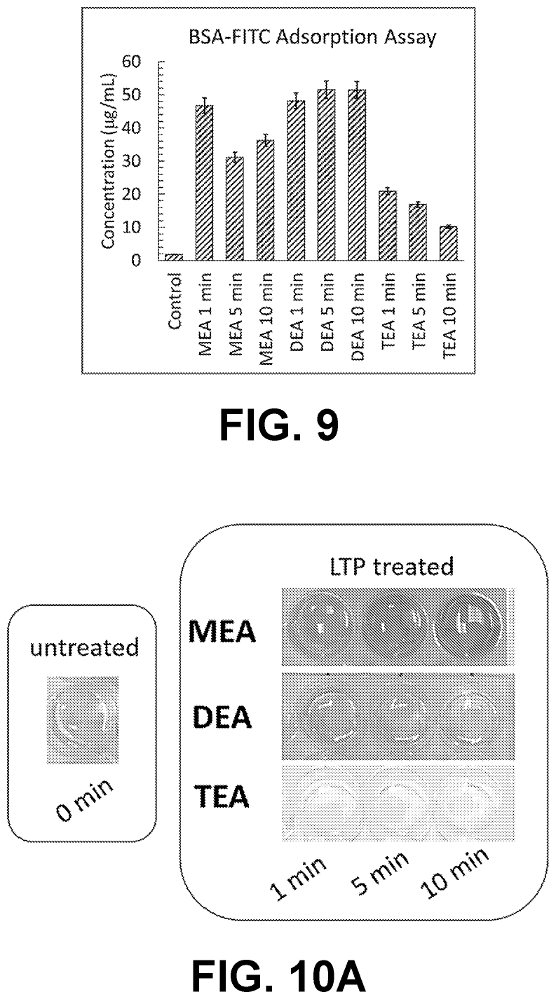

FIG. 9 illustrates BSA-FITC adsorption tests comparing the untreated controls to the various MEA, DEA and TEA LTP treated samples for 1, 5, and 10 minute exposure times. The bars are the standard error from at least 100-150 separate pixel measurements from triplet group trials of the fluorescent analysis in ImageJ. The values are the numerical average.

Nitric oxide (NO) release measurements. A standard Griess assay procedure adopted from Promega was conducted with sourced components and measured with a BioRad 580 absorbance plate reader. Samples were incubated at various time points to elucidate the released NO profiles in 1× PBS at RT. 100 μL of test sample was added to 100 μL of Griess reagent solution and incubated for 10 min at room temperature (RT) to an end point measured with dual-wavelength mode of 415 nm monitor and 530 nm measure filters on the plate reader. Samples were replicated in triplicate.

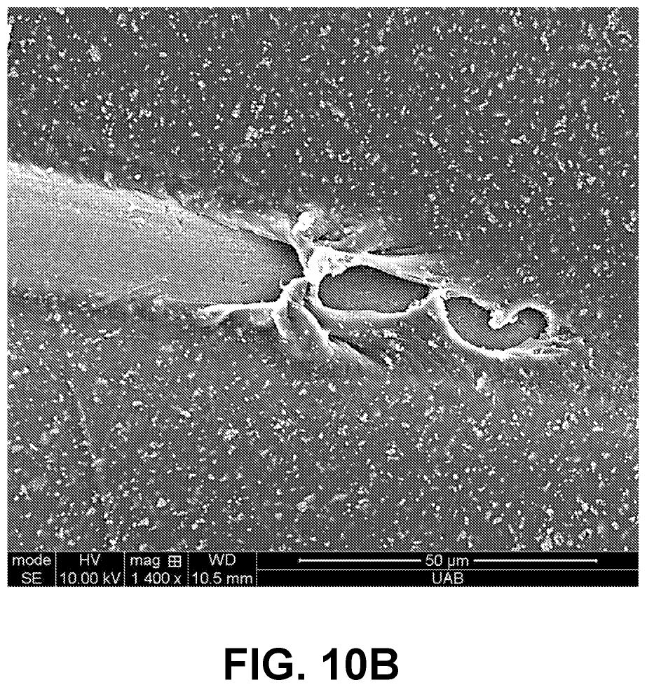

FIG. 10A illustrates examples of spot test for NO release using the Griess assay. The comparisons of untreated control vs. 1 min, 5 min, and 10 min LTP exposure times for MEA, DEA and TEA are shown. The orange color in the MEA 1 min could be accounted for by undesirable side products. FIG. 10B is a SEM image showing an example of SEM of an implant composite coating silica-DEA or dopamine coating.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

The term “substantially” is meant to permit deviations from the descriptive term that don't negatively impact the intended purpose. Descriptive terms are implicitly understood to be modified by the word substantially, even if the term is not explicitly modified by the word substantially.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. The term “about” can include traditional rounding according to significant figures of numerical values. In addition, the phrase “about ‘x’ to ‘y’” includes “about ‘x’ to about ‘y’”.

1. A bio-compatible implant or device, comprising:
a metallic structure; and
an organosilane plasma polymerization (OPP) coating disposed on a surface of the metallic structure, the OPP coating comprising inorganic silica disposed on bare metal of the metallic structure and forming a nano-textured surface.

2. The bio-compatible implant or device of claim 1, wherein the metallic structure is a stent, wire or catheter.

3. The bio-compatible implant or device of claim 1, wherein the metallic structure comprises nitinol (NiTi), stainless steel or cobalt chrome alloy.

4. The bio-compatible implant or device of claim 1, wherein the OPP coating is a SiOₓ-like coating disposed on the surface of the metallic structure.

5. The bio-compatible implant or device of claim 4, wherein the OPP coating is disposed on the surface by plasma treatment over a period of approximately 30 minutes.

6. The bio-compatible implant or device of claim 5, wherein the surface of the metallic structure is pretreated prior to the plasma treatment.

7. The bio-compatible implant or device of claim 4, wherein the OPP coating has a thickness in a range from about 50 nm to about 1 micron.

8. A bio-compatible implant or device, comprising:
a metallic structure; and
a composite coating disposed on a surface of the metallic structure, the composite coating comprising silica-DEA, silica-MEA, or silica-TEA functionalized coating disposed on bare metal of the metallic structure and forming a nano-textured surface.

9. The bio-compatible implant or device of claim 8, wherein the composite coating comprises silica-DEA, dopamine, dopamine acrylic derivatives or dopamine acrylamide polymerized/co-polymerized coating disposed on the bare metal of the metallic structure.

10. The bio-compatible implant or device of claim 8, wherein the composite coating comprises silica-allyl amine, L-lysine, or tyrosine, polymerized/co-polymerized coating disposed on the bare metal of the metallic structure.

11. The bio-compatible implant or device of claim 8, wherein the metallic structure is a stent, wire or catheter.

12. The bio-compatible implant or device of claim 8, wherein the metallic structure comprises nitinol (NiTi), stainless steel or cobalt chrome alloy.

13. The bio-compatible implant or device of claim 8, wherein the composite coating is disposed on the surface by plasma treatment.

14. The bio-compatible implant or device of claim 13, wherein the composite coating is disposed over a period of approximately 30 minutes.

15. The bio-compatible implant or device of claim 13, wherein the surface of the metallic structure is pretreated prior to the plasma treatment.

16. The bio-compatible implant or device of claim 8, wherein the composite coating has a thickness in a range from about 50 nm to about 1 micron.

17. A method for preparing a bio-compatible implant or device, comprising:
providing a metallic structure; and
exposing the metallic structure to organosilane plasma polymerization (OPP) process to form an OPP coating on a surface of the metallic structure to form the bio-compatible implant or device, where the OPP coating comprises inorganic silica disposed on bare metal of the metallic structure thereby forming a nano-textured surface.

18. The method of claim 17, wherein the OPP coating is a composite coating comprising silica-DEA, silica-MEA, or silica-TEA functionalized coating.

19. The method of claim 17, wherein the OPP coating is disposed on the surface by plasma treatment over a period of approximately 30 minutes.

20. The method of claim 17, wherein the OPP coating has a thickness in a range from about 50 nm to about 1 micron.
